(19) United States
(12) Patent Application Publication
(10) Pub. No.: US 2023/0201072 A1
(43) Pub. Date: Jun. 29, 2023

(54) ELECTRO-MECHANICAL SEXUAL STIMULATION DEVICE

(71) Applicant: Michael Haggerty, Cape coral, FL (US)

(72) Inventor: Michael Haggerty, Cape coral, FL (US)

(21) Appl. No.: 18/091,085

(22) Filed: Dec. 29, 2022

Related U.S. Application Data
(60) Provisional application No. 63/294,695, filed on Dec. 29, 2021.

(51) Int. Cl. A61H 19/00

(52) U.S. Cl. CPC A61H 19/44; A61H 19/32; A61H 19/34; A61H 2201/1685; A61H 2201/1215

(57) ABSTRACT

A sexual stimulation device for applying stimulus to an erogenous zone of the human body. The sexual stimulation device has a body and an interchangeable head that can be interchangeably coupled to the body. The body has a motor. The interchangeable head has a stimulation surface. A kit is also provided having two such bodies and four interchangeable heads. Each of the four interchangeable heads has a different stimulation surface.

### CROSS-REFERENCE TO RELATED APPLICATIONS

This application claims priority from a U.S. Provisional Patent Appl. No. 63/294,695 filed on Dec. 29, 2021, which is incorporated herein by reference in its entirety.

### FIELD OF INVENTION

The present invention relates to a sexual stimulation device, and more particularly, the present invention relates to an electro-mechanical stimulation device with interchangeable heads and a kit having the electro-mechanical stimulation devices and the interchangeable heads for use by partners.

### BACKGROUND

Sexual stimulation devices are well-known in the art and are becoming quite popular. Sexual stimulation devices are used to stimulate sensitive erogenous zones in a body. The sexual stimulation devices generally have a head with a plain surface or a surface with multiple protuberances which when rubbed in a massaging action against a sensitive tissue result in excitation. For example, vibrators are known that are applied directly to exposed sensitive tissues, such as the clitoris for stimulation. Dildos are known that are inserted into vaginal cavities for stimulation. Both mechanical and electronic stimulation aids are available, wherein the mechanical stimulation aid can be operated by hand while the electronic stimulation aid includes an electronic motor.

The known sexual stimulation devices, however, suffer from a variety of drawbacks and limitations. Generally, sexual stimulation devices are designed for stimulating female-sensitive erogenous zones only. The known sexual stimulation devices are for self-pleasure which limits the use and popularity of the sexual stimulation devices. Also, frequent application of the same stimulation device results in habituation effects or conditioning of the erogenous zone. Although a variety of stimulation devices are available, the use of different stimulation devices is costly.

A need is therefore desired for a versatile sexual stimulation device and a kit that has the advantage of distinct stimulation devices and overcomes the aforesaid drawbacks with conventional sexual stimulation devices.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a sexual stimulation device with interchangeable heads, and a kit comprising the sexual stimulation devices and the interchangeable heads.

It is another object of the present invention that the sexual stimulation device and the kit are economical to manufacture and use.

It is still another object of the present invention that the sexual stimulation device and the kit can be used by partners in sexual activity.

It is a further object of the present invention that the sexual stimulation device and the kit adds excitement and fun.

It is still a further object of the present invention that the different stimulation heads can be interchangeably used for stimulation of different erogenous zones.

It is an additional object of the present invention that the sexual stimulation device can be adapted for use by both males and females.

In one aspect, disclosed is a sexual stimulation device comprising a body, the body comprises a housing of an elongated cylindrical profile, a motor encased with the housing, and a coupler operably coupled to the motor; and an interchangeable head, the interchangeable head configured to be interchangeably coupled to the body through the coupler, the interchangeable head comprises a stimulation surface configured to apply a stimulus to an erogenous zone of a human body. The sexual stimulation device further comprises a battery also encased within the housing, the battery configured to power the motor. In one case, the interchangeable head is half-egg shaped and the stimulation surface has protuberances dispersed throughout the stimulation surface. In another aspect, the interchangeable head is of an elongated profile and resembles a human penis. In other aspect, the interchangeable head is of an elongated cylindrical profile and the stimulation surface has protuberances dispersed throughout an area of the interchangeable head. In another aspect, the interchangeable head is of an elongated cylindrical profile having a cavity, and the stimulation surface of the cavity has protuberances dispersed throughout the stimulation surface.

In one aspect, disclosed is a kit for sexual stimulation comprising a first body and a second body, wherein each of the first body and the second body comprises a housing of an elongated cylindrical profile, a motor encased with the housing, and a coupler operably coupled to the motor; and four interchangeable heads, each of the four interchangeable heads configured to interchangeably couple to the first body or the second body through the respective coupler, each of the four interchangeable heads comprises a stimulation surface configured to apply a stimulus to an erogenous zone of a human body, wherein each of the four interchangeable heads has a different stimulation surface. The first body has a vibratory motor, and the second device has a rotary motor. Each of the first body and the second body further comprises a battery encased within the housing, the battery configured to power the motor. A first interchangeable head of the four interchangeable heads is half-egg shaped and protuberances dispersed throughout the respective stimulation surface. A second interchangeable head of the four interchangeable heads is of an elongated profile and resembles a human penis. A third interchangeable head of the four interchangeable heads is of an elongated cylindrical profile and protuberances dispersed throughout the respective stimulation surface. A fourth interchangeable head of the four interchangeable heads is of an elongated cylindrical profile having a cavity, the cavity has the stimulation surface, and protuberances dispersed throughout the respective stimulation surface.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a perspective view of the sexual stimulation device having a body and an interchangeable head, according to an exemplary embodiment of the present invention.

FIG. 2 shows an interchangeable head of a penis shape, according to an exemplary embodiment of the present invention.

FIG. 3 shows another interchangeable head with an elongated profile and several protuberances dispersed throughout the outer surface of the interchangeable head, according to an exemplary embodiment of the present invention.

FIG. 4 shows another interchangeable head having a cavity with several protuberances disposed over an outer surface of the cavity, according to an exemplary embodiment of the present invention

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an” and “the” are intended to include the plural forms as well, unless the context indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

The following detailed description is described with reference to the drawings, wherein like reference numerals are used to refer to like elements throughout. In the following description, for purposes of explanation, specific details may be outlined in order to provide a thorough understanding of the subject innovation. It may be evident, however, that the claimed subject matter may be practiced without these specific details. In other instances, well-known structures and apparatus are shown in block diagram form in order to facilitate describing the subject innovation. Moreover, the drawings may not be to scale.

Disclosed is a sexual stimulation device with interchangeable heads, wherein the head includes a stimulation surface for applying stimulus to an erogenous zone. Different heads can include different types of stimulation surfaces for stimulating different erogenous zones in a body or applying a different stimulus to the same erogenous zone. The interchangeable heads can be provided for stimulating erogenous zones of males and females. Thus, the same sexual stimulation device can be used by both males and females by changing the heads.

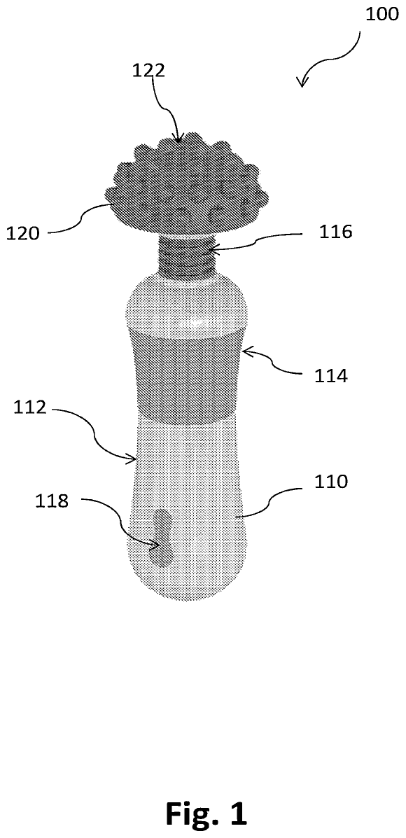

Referring FIG. 1 which shows an exemplary embodiment of the disclosed sexual stimulation device 100 that includes a body 110 and a head 120. The body 110 has a housing 112 of an elongated and ergonomic profile. The housing allows the disclosed sexual stimulation device 100 to be held in a hand for applying stimulus by the disclosed sexual stimulation device. As shown in the drawings, the housing can be of a cylindrical shape that tapers down slightly in the middle for the ergonomic holding of the device in hand. Grip 114 can be disposed around the housing for comfortably holding the device, wherein the grip provides cushioning for the fingers and prevents them from slipping. The grip can be made from any rubbery material, such as silicone. The outer surface of the housing can be smooth for making cleaning the device easier. Moreover, the outer surface can be made glossy for an aesthetic appearance. Suitable combinations of colors can be used, such as white, blue, and pink for an aesthetic appearance. The housing can be off-white while the sleeve can be a blue color. The coupler 116 and interchangeable heads 120 can be pink. It is obvious that the colors may be optional, and any color can be used for any component without departing from the scope of the present invention. Moreover, the outer surface of the body can be matt finish or grainy, and any modification of the outer surface of the body is within the scope of the present invention. Also, the housing can be made airtight as well as watertight to prevent the ingress of dust and moisture.

The body can also include a motor for driving the head. More than one motor can be provided for moving including vibratory and/or rotary motion. The device can be switched between vibratory and rotary motion. For example, a switch that protrudes from the housing can be provided, wherein the switch can be operated for switching between the vibratory and rotary motion. The switch can also be used to power on and off the disclosed device. An option to control the speed of either motor can also be provided. For example, a knob may protrude from the housing, wherein the knob can be turned to increase or decrease the speed of the functional motor. Alternatively, a press button can also be used in place of a knob wherein the button can be pressed to increase or decrease the speed of the motor.

One or more motors can be encased within the housing which can be sealed to prevent ingress of any water that may harm the motor. The motor can be powered by a battery also encased within the housing. The battery can be a disposable battery or a rechargeable battery. Preferably, a rechargeable battery can be used, such as a lithium-ion battery. In the case of a rechargeable battery, a charging circuitry can also be provided which can also be encased within the housing and operably coupled to the battery. The charging circuitry may have an interface for connecting to the external power supply. The charging port may be accessible from outside the housing, that can be connected to an external power adaptor for charging the battery. A switch 118 for turning the motor on and off, and also changing the speed of the motor is shown.

The motor can be operably coupled to a coupler 116. The coupler allows for coupling the interchangeable head to the motor. The coupler may include an interlocking mechanism for securing the head to the body.

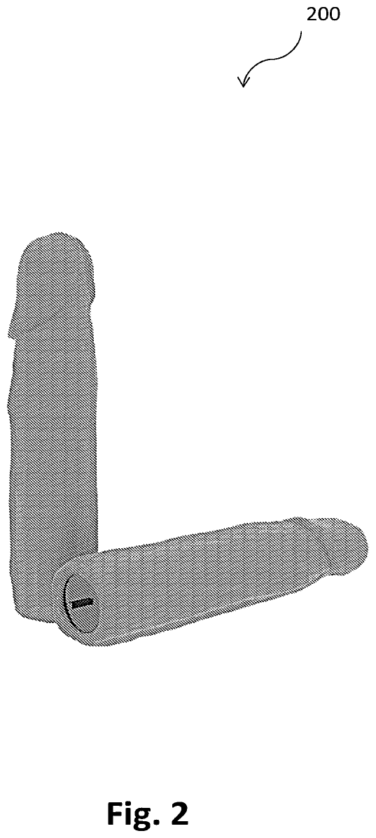

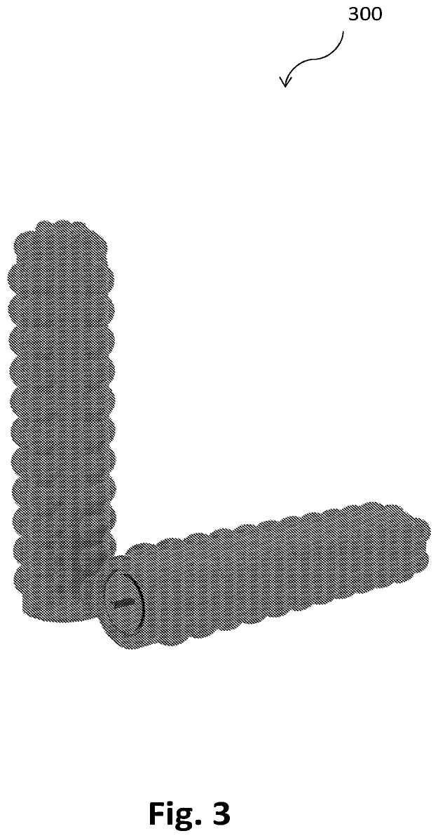

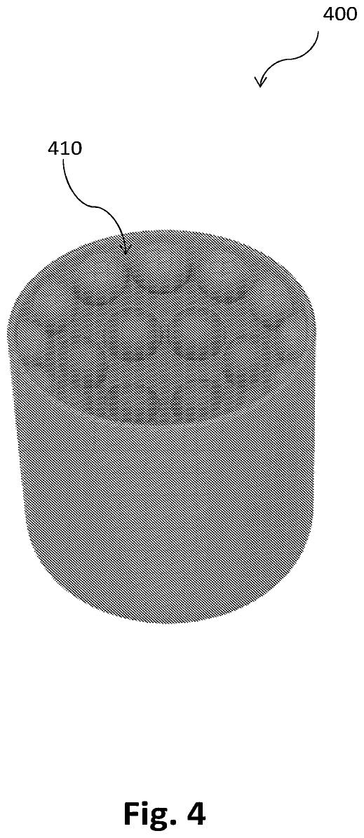

The interchangeable head 120 shown in FIG. 1 is half egg-shaped with multiple protrusions 122 on the stimulation surface thereof. This can be used to stimulate the clitoris and the like erogenous zones. FIG. 2 illustrates another embodiment of the interchangeable head 200 which is of a penis shape. FIG. 3 shows an interchangeable head 300 of an elongated profile that can be inserted into the vaginal cavity for stimulation. The stimulation surface of the interchangeable head 300 has several stimulation protuberances dispersed throughout the stimulation area. FIG. 4 shows the interchangeable head 400 that has a cavity 410 and can be used to stimulate the male erogenous zones.

Also, disclosed is a kit that can include two bodies of sexual stimulation devices. Both bodies can have the same motors or different ones. For example, one stimulation device can have a vibratory mechanism while the other can have a rotary mechanism. The four interchangeable heads described above can also be included in the kit.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A sexual stimulation device comprising:
a body, the body comprises:
a housing of an elongated cylindrical profile,
a motor encased with the housing, and
a coupler operably coupled to the motor; and
an interchangeable head, the interchangeable head configured to be interchangeably coupled to the body through the coupler, the interchangeable head comprises a stimulation surface configured to apply a stimulus to an erogenous zone of a human body.

2. The sexual stimulation device of claim 1, wherein the sexual stimulation device further comprises:
a battery also encased within the housing, the battery configured to power the motor.

3. The sexual stimulation device according to claim 1, wherein the interchangeable head is half-egg shaped and the stimulation surface has protuberances dispersed throughout the stimulation surface.

4. The sexual stimulation device according to claim 1, wherein the interchangeable head is of an elongated profile and resembles a human penis.

5. The sexual stimulation device according to claim 1, wherein the interchangeable head is of an elongated cylindrical profile and the stimulation surface has protuberances dispersed throughout an area of the interchangeable head.

6. The sexual stimulation device according to claim 1, wherein the interchangeable head is of an elongated cylindrical profile having a cavity, and the stimulation surface of the cavity has protuberances dispersed throughout the stimulation surface.

7. A kit for sexual stimulation comprising:
a first body and a second body, wherein each of the first body and the second body comprises:
a housing of an elongated cylindrical profile,
a motor encased with the housing, and
a coupler operably coupled to the motor; and
four interchangeable heads, each of the four interchangeable heads configured to interchangeably couple to the first body or the second body through the respective coupler, each of the four interchangeable heads comprises a stimulation surface configured to apply a stimulus to an erogenous zone of a human body, wherein each of the four interchangeable heads has a different stimulation surface.

8. The kit according to claim 7, wherein the first body has a vibratory motor, and the second device has a rotary motor.

9. The kit according to claim 7, wherein each of the first body and the second body further comprises:
a battery encased within the housing, the battery configured to power the motor.

10. The kit according to claim 7, wherein a first interchangeable head of the four interchangeable heads is half-egg shaped and protuberances dispersed throughout the respective stimulation surface.

11. The kit according to claim 10, wherein a second interchangeable head of the four interchangeable heads is of an elongated profile and resembles a human penis.

12. The kit according to claim 11, wherein a third interchangeable head of the four interchangeable heads is of an elongated cylindrical profile and protuberances dispersed throughout the respective stimulation surface.

13. The kit according to claim 12, wherein a fourth interchangeable head of the four interchangeable heads is of an elongated cylindrical profile having a cavity, the cavity has the stimulation surface, and protuberances dispersed throughout the respective stimulation surface.
